(19) United States
(12) Patent Application Publication
(10) Pub. No.: US 2026/0228956 A1
(43) Pub. Date: Aug. 6, 2026

(54) COMPUTER-IMPLEMENTED METHOD FOR PROVIDING AT LEAST ONE TWO-DIMENSIONAL UNFOLDED IMAGE DATASET

(71) Applicant: Siemens Healthineers AG, Forchheim (DE)

(72) Inventors: Dominik FRIEDMANN, Erlangen (DE); Felix LADES, Erlangen (DE); Michael SUEHLING, Erlangen (DE); Eva EIDENBERGER, Nuernberg (DE); Andrei CHEKKOURY, Erlangen (DE)

(73) Assignee: Siemens Healthineers AG, Forchheim (DE)

(21) Appl. No.: 19/463,449

(22) Filed: Jan. 29, 2026

(30) Foreign Application Priority Data
Jan. 31, 2025 (EP) 25155288.1

(51) Int. Cl. G06T 15/00; G06T 7/11; G06T 7/73

(52) U.S. Cl. CPC G06T 15/00; G06T 7/11; G06T 7/75; G06T 2200/04; G06T 2200/24; G06T 2207/30004; G06T 2210/41

(57) ABSTRACT

A computer-implemented method comprises: provisioning a first three-dimensional image dataset; determining a set of characteristic structure points of the anatomical structure; provisioning a two-dimensional model of the anatomical structure; determining a set of characteristic specimen points by mapping the set of structure points to the model; determining a deformation surface in a plane with the set of specimen points; calculating a first deformed deformation surface by applying a deformation algorithm to the deformation surface, the set of structure points and the set of surface points; calculating a set of voxel positions relative to the first three-dimensional image dataset or a provided second three-dimensional image dataset and based on the first deformed deformation surface; calculating the unfolded 2D image dataset of the anatomical structure based on the first 3D image dataset or the second 3D image dataset and the set of voxel positions; and provisioning the unfolded 2D image dataset.

### CROSS-REFERENCE TO RELATED APPLICATION(S)

The present application claims priority under 35 U.S.C. § 119 to European Patent Application No. 25155288.1, filed Jan. 31, 2025, the entire contents of which are incorporated herein by reference.

### FIELD

One or more example embodiments of the present invention relate to a computer-implemented method for providing at least one two-dimensional unfolded image dataset, in particular at least of a part of the hand, on the basis of a three-dimensional image dataset. Furthermore, one or more example embodiments of the present invention relate to a corresponding data processing system, a system comprising a medical imaging system and such a data processing system, as well as a computer program product and a computer-readable storage medium.

### BACKGROUND

Independent of the grammatical term usage, individuals with male, female or other gender identities are included within the term.

Not only but in particular in the case of complex accidents it is crucial to obtain a quick overview of the overall effects on a patient's body, e.g. in order to find out whether and where a patient has fractures or dislocations in the bones, or smaller vascular injuries. In such cases three-dimensional images are often made, for example via a computed tomography (CT) scan. In the case of curved anatomical structures or those which can assume arbitrary positions and postures in the image datasets of such a scan, such as for example the hands, it is often difficult to assess the full extent of damage using conventional methods. Scrolling, for example, through the axial sections of a three-dimensional image dataset, is very time-consuming, and most of the time bone fractures or vascular injuries extend over more than one cross-sectional image, which prevents the entire three-dimensional extent of the fracture or injury from being visible easily and in one view. In contrast, three-dimensional overview images such as volume rendering techniques (VRT) visualize in particular the surface of structures and only from a certain perspective. The use of these methods can consequently be tedious and/or prone to fractures or vascular injuries being overlooked. Not only can this lead to negative outcomes for the patient, but it can also have financial and legal consequences.

One possibility for an improved visualization of such anatomical structures may be the generation of an unfolded image dataset, which converts the anatomical structure into a two-dimensional representation on the basis of the three-dimensional image dataset. For example, EP 3828836 B1 discloses a method for unfolding a tubular structure, for example a blood vessel. In DE102011085860B4 a method for medical imaging of a part of the body, in particular the hand, is disclosed, wherein the image content of a curved surface is mapped to a viewing plane.

### SUMMARY

An object of one or more example embodiments of the present invention is to specify an improved method comprising the provision of an unfolded two-dimensional image dataset of an anatomical structure.

At least this object is inventively achieved by the subject matter of the independent claims. Advantageous forms of embodiment with expedient developments are the subject matter of the subclaims.

One or more example embodiments of the present invention relate to a computer-implemented method for providing at least one two-dimensional unfolded image dataset (unfolded 2D image dataset) of an anatomical structure, in particular at least of a partial area of the hand. In a first step a first three-dimensional image dataset (3D image dataset) which includes the anatomical structure is provided. In a further step a set of characteristic structure points of the anatomical structure is determined on the basis of the first 3D image dataset. In a further step a two-dimensional model of the anatomical structure is provided. In a further step a set of characteristic specimen points is determined by mapping the set of structure points to the model. In a further step a deformation surface is determined in a plane with the set of specimen points, wherein each specimen point of the set of specimen points is represented by a surface point in the deformation surface. In a further step a first deformed deformation surface is determined by applying a deformation algorithm to the deformation surface, the set of characteristic structure points and the set of surface points, wherein each surface point from the set of surface points is moved to the corresponding characteristic structure point from the set of characteristic structure points. In a further step a set of voxel positions is calculated in relation to the first 3D image dataset or in relation to a provided second 3D image dataset and on the basis of the first deformed deformation surface. In a further step the unfolded 2D image dataset of the anatomical structure is calculated on the basis of the first 3D image dataset or the second 3D image dataset and the set of voxel positions. In a further step the unfolded 2D image dataset is provided.

In this case the steps of the proposed method described above can be carried out one after the other and/or at least partially simultaneously. Further, the steps of the proposed method can be at least partially, in particular completely, computer-implemented.

The anatomical structure can be an anatomical structure of a human and/or animal patient. However, it can also comprise an examination phantom. In particular, the anatomical structure can comprise such structures, whose variance in their formation within the expected patient population enables a pattern-based approach, i.e. whose formation within the expected patient population shows a sufficient correspondence. The anatomical structure can for example comprise a sub-region of the hand, the arm or the foot. It can for example comprise part of a patient's skeletal structure. It can also comprise other anatomical structures. The anatomical structure can comprise multiple sub-structures, also called sub-units. For example, if the method relates to a hand, the sub-structures can respectively comprise the fingers or at least all phalanges pertaining to a finger. Particularly advantageously, the method can for example be employed in the region of the hands, since there the anatomy has sufficiently large correspondences between patients, but the location and position can vary particularly markedly in 3D image datasets. An image dataset, which maps the anatomical structure in an unfolded representation, can here enable an advantageously improved representation and diagnosis.

The provision of a first or also of a provided second 3D image dataset can in particular comprise the capture and/or reading of a computer-readable data store and/or the receipt from a data storage unit, for example a database. Further, the image dataset can be provided by a processing unit of a medical imaging device for the acquisition of the 3D image dataset. The medical imaging device can for example comprise a computed tomography system (CT) and/or a magnetic resonance tomography system (MRT) and/or a medical X-ray device, in particular a medical C-arm X-ray device, and/or an ultrasound device and/or a positron emission tomography system (PET). The provision of a 3D image dataset can also comprise the generation, in particular the reconstruction, of the 3D image dataset on the basis of pre-captured measured data.

A 3D image dataset permits a three-dimensional, in particular spatially three-dimensional, representation of an imaging area. In the proposed method the imaging area comprises at least the anatomical structure. A 3D image dataset can also be represented as a plurality of slice image datasets, so-called cross-sectional representations. A slice image dataset in each case comprises a slice of the 3D image dataset at a position along a marked axis. A slice image dataset in each case allows a two-dimensional, in particular spatially two-dimensional, representation of the respective slice of the 3D image dataset.

Such a 3D image dataset advantageously comprises multiple voxels, in particular image points. In this case each voxel can preferably have an image value in each case, also referred to below as a voxel value, in particular a CT image value in HU (“Hounsfield Units”) in the case of a CT scan, or an intensity value analogous thereto. Normally the voxel centers are arranged in a regular three-dimensional lattice. Analogously to this, a slice image dataset can comprise multiple pixels, in particular image points. In this case each pixel can preferably have an image value in each case, in particular a CT image value in HU (“Hounsfield Units”) in the case of a CT scan, or an intensity value analogous thereto.

The unfolded 2D image dataset in particular has multiple pixels, in particular image points, wherein each pixel preferably has an image value in each case, in particular a CT image value in HU (“Hounsfield Units”) in the case of a CT scan, or an intensity value analogous thereto. In the proposed method the image values of the unfolded 2D image dataset are based on the set of previously calculated voxel positions of the first or second 3D image dataset and the image values of the corresponding 3D image dataset.

The set of the characteristic structure points in the first 3D image dataset can for example be selected (semi-)automatically and/or on the basis of a corresponding manual user input. The set of characteristic structure points can comprise one or a plurality of discrete points and/or multiple points which comprise a one-dimensional line, which can be curved in three dimensions. They can also form a two-dimensional surface or multiplicity, which can be flat or curved in three dimensions. The unfolded 2D image dataset enables the determined characteristic structure points which in the 3D image dataset are where appropriate arranged on a curved line or surface, to be represented in a single two-dimensional mapping. The characteristic structure points can consequently span the image plane of interest for the unfolded 2D image dataset. In particular, the set of characteristic structure points can map the three-dimensional course and/or location of the anatomical structure in the 3D image dataset, in particular also in a reduced form.

In particular, the set of the characteristic structure points in the first 3D image dataset can be based on the application of a segmentation algorithm and/or edge detection and/or a center line extraction algorithm to the first 3D image dataset with regard to the anatomical structure. If the anatomical structure for example comprises at least one sub-region of the hand, the set of the characteristic structure points can for example for at least one or also multiple or all fingers, in particular its or their associated phalanges, comprise at least one characteristic structure point, but advantageously a plurality of characteristic structure points, which maps the location and/or course of the finger or fingers in the 3D image dataset. For example, the at least one, but advantageously the plurality of characteristic structure points can be located on a center line, also referred to as a center axis, of this finger or the center lines of the respective fingers, in particular of the phalanges pertaining to the finger. The same can also be transferred to a mapped foot or arm or other anatomical structures. In addition there can also be other embodiments.

Determination of the set of characteristic structure points can also comprise determining additional information on the characteristic structure points, for example annotation information, for instance the affiliation of the characteristic structure points to anatomical sub-structures, or anatomical landmarks. If for example the anatomical structure comprises a sub-region of the skeleton, the characteristic structure points can in each case be determined as assigned to a determined part of the skeleton. The structure points can be “labeled”.

The provision of a 2D model, also referred to as a template, of the anatomical structure can in particular comprise a capture and/or reading of a computer-readable data store and/or a receipt from a data storage unit, for example a database. The model can in particular map an average anatomy of the anatomical structure in a patient population averagely affected by the method or in an expected patient population. It can thus comprise a standardized representation of the anatomical structure. For example, the model can be created by averaging anatomical structures from a large random sample of test subjects. In particular, the model can be tuned to the set of characteristic structure points to be determined. The model can represent the image plane, spanned by the determined characteristic structure points, through the anatomical structure in a standardized form. The model can in particular correspond to a two-dimensional representation of the anatomy of the anatomical structure, in particular when the anatomical structure is arranged along a plane. If for example the method relates to the hand or at least a sub-region of the hand, the 2D model can map the anatomy of a hand, in particular the arrangement of the bones of the hand, in a standardized form. The model can also map the anatomy of the anatomical structure in a reduced form. If for example the characteristic structure points comprise a center axis of the anatomical structure, the model can likewise comprise a provided specimen center axis of an anatomical specimen structure. Multiple models can also be provided. For example, the patient population can be divided into categories on the basis of patient information, for instance with regard to age or sex. For example, a corresponding model could then be selected manually or automatically on the basis of available patient data for the present case. In particular, the model can comprise further data over and above a straightforward mapping of the anatomy, for example annotation information such as for instance identification markings of anatomical sub-structures or anatomical landmarks.

When determining the set of characteristic specimen points a counterpart to a characteristic structure point at an identical relative position inside the anatomical structure can in particular be determined in the model in each case. Each specimen point of the set of specimen points can be assigned to a characteristic structure point of the set of structure points. The number of the set of specimen points and the number of the set of characteristic structure points can correspond 1:1 to each other, i.e. each structure point can be assigned a specimen point in a 1:1 ratio.

The deformation surface can in particular have a smooth geometry and/or can comprise a (surface) lattice, in particular a regular lattice. The lattice can for example comprise a two-dimensional arrangement of nodes, in particular m x n nodes, where m and n are whole numbers. The deformation surface can extend at least over the entire anatomical structure and/or the arrangement of the determined set of specimen points. It can extend over the entire image area of the first 3D image dataset or of the model. In particular, the deformation surface can be aligned with the model.

The deformation surface is in particular intended such that the set of specimen points is arranged in the plane of the deformation surface, wherein each specimen point is represented by a surface point in the deformation surface. The specimen points of the set of specimen points in the deformation surface and the surface points of the set of surface points can be identical. A surface point can consequently be given directly by a specimen point in the deformation surface. If for example the deformation surface is intended as a lattice, a surface point can in each case correspond to the lattice node of the lattice nearest to a specimen point. The set of surface points can consequently correspond to a set of lattice node points, which in each case are nearest to the specimen points of the set of specimen points in the deformation surface. There may also be other embodiments.

Applying the deformation algorithm to the initial smooth deformation surface results in a deformed deformation surface, generally curved in all three dimensions, by which the characteristic structure points are comprised. The deformation algorithm can for example be based on an “As-Rigid-As-Possible” algorithm. Examples of this are described in Sorkine, O., et al (As-Rigid-As-Possible Surface Modeling. EUROGRAPHICS/ACM SIGGRAPH Symposium on Geometry Processing (2007), 109-116). In particular, the Laplace operator of the lattice can be preserved during the deformation, meaning that the edge lengths and angles between the edges of the lattice are preserved. The deformation algorithm can for example comprise a nonlinear and/or iterative optimization. A smooth deformation of the planar lattice can advantageously be ensured. In this case the structure points or surface points serve as what are known as constraint points for the deformation of the initial, undeformed deformation surface via the deformation algorithm, in order to preserve a deformed deformation surface which comprises the characteristic structure points.

After the deformation the voxel positions can be determined, in particular voxel positions which correspond to the pixels of the unfolded 2D image dataset. The subset of the plurality of voxels which correspond to the pixels of the unfolded 2D image dataset can for example be determined by determining an intersection of the two-dimensional deformed deformation surface in three-dimensional space with the plurality of voxels of the three-dimensional representation of the first or of a provided second 3D image dataset. The subset of the plurality of voxels can consist of all voxels of the plurality of voxels which can be touched or intersected by the two-dimensional deformed deformation surface, or lie in a predefined neighborhood of the said touched or intersected voxels. However, there can be other methods of transmission. The two-dimensional unfolded image, in particular the image values of the pixels of the unfolded 2D image dataset, can then be scanned, i.e. calculated, by extracting voxel values from the voxel positions in the first or second 3D image dataset. As a result, a bidirectional assignment between 3D positions in the first or second 3D image dataset and 2D positions in the unfolded 2D image dataset can be created. Determining the voxel positions or the image values extracted therefrom can comprise an interpolation, since a deformed deformation surface does not necessarily run through the voxel centers of a 3D image dataset, which are normally arranged in a fixed grid.

A provided second 3D image dataset can in this case be an image dataset which is based on measured data from the same acquisition procedure as the first 3D image dataset and thus the location and position of the anatomical structure in the second 3D image dataset are unchanged relative to the first 3D image dataset. The second 3D image dataset can represent the same imaging area as the first 3D image dataset. However, the second 3D image dataset can differ from the first 3D image dataset for example with regard to a reconstructed image resolution or an information content, for example relating to spectral image information. If the second 3D image dataset differs from the first 3D image dataset with regard to an image resolution, in order to calculate the voxel positions and the unfolded image dataset it can for example be provided that an edge length of the polygons of a planar surface lattice representing the deformation surface and/or a number of the points scanned per polygon is adjusted in order to adjust the resolution of the resulting unfolded 2D image dataset to the resolution of the second 3D image dataset. For an unfolding of the second 3D image dataset, a renewed application of the deformation algorithm can advantageously be dispensed with and thus an unfolded image dataset can be determined in a computationally efficient and time-efficient manner.

The provision in particular of the unfolded 2D image dataset can for example comprise writing the data to a computer-readable storage unit or displaying it on a display unit, for example on a monitor. It can also comprise providing the data via a suitable interface for a subordinate processing unit, which is designed for further processing of the unfolded 2D image dataset.

The provision of the unfolded 2D image dataset makes it possible to obtain a two-dimensional representation of complexly arranged and shaped anatomical structures from the 3D image dataset, which enables a detailed examination of the anatomical structure and the spatial relationships of individual sub-structures to one another in a single view. In particular when the characteristic structure points are chosen appropriately, this representation can in particular represent a two-dimensional mapping of the anatomical structure if the anatomical structure had been arranged along a plane during data acquisition. The pattern-based approach in this case enables a particularly efficient, robust and simple implementation of the deformation algorithm through the constraint points comprised thereby for the deformation of the initial smooth deformation surface. The simple provision and assignment of annotation information on the basis of the model can advantageously also facilitate automatic reporting of medical findings.

In accordance with one aspect of the proposed method the determination of the set of characteristic structure points can comprise the segmentation of the anatomical structure, wherein the set of structure points is determined on the basis of the segmented structure.

The segmentation can for example be achieved in a pixel-based, voxel-based, edge-based, surface-based and/or region-based manner. The segmentation can also be based on a model-based method, wherein assumptions are made about the object to be segmented. The segmentation can be implemented by the computing unit on an automated or semi-automated basis. For example, starting points or nuclei or rough contour information for the segmentation can be set manually. However, in a preferred variant a machine-learning method, i.e. a trained function, is employed for the segmentation of the anatomical structure. In particular, a neural network can be employed in the form of a so-called U-Net (see for example: Ronneberger O et al. U-Net: Convolutional Networks for Biomedical Image Segmentation. https://doi.org/10.48550/arXiv.1505.04597), in particular a so-called nnU-Net (see for example: Isensee F et al. nnU-Net: Self-adapting Framework for U-Net-Based Medical Image Segmentation. https://doi.org/10.48550/arXiv.1809.10486). However, other architectures known from the prior art can also be used, in particular those based on a deep learning model with an encoder-decoder architecture.

The segmentation can include the entire anatomical structure being segmented. It can include sub-structures of the anatomical structure being segmented separately in each case. For example, there can be a bone-by-bone segmentation if the anatomical structure comprises part of the skeleton. For example, each finger, in particular all phalanges pertaining to a finger, of a hand can be segmented individually. Furthermore, it can include the anatomical structure being segmented in a reduced form. For example, only one sub-volume of the anatomical structure may be segmented. This can for example in each case include a sub-volume around a center axis of a bone structure, for instance of a respective finger of a hand. In particular, when using a trained function, the result of the segmentation depends on the training data used. If the training data in each case only comprises a sub-volume of the anatomical structure as comparative data, the result of the segmentation based on the trained function will be available accordingly. The same applies to a different result of the segmentation. Based on a segmented structure, a set of characteristic structure points can be determined in an advantageously simplified and robust manner, in particular also automatically.

Furthermore, it can be provided that a segmentation is accompanied by a determination of further information, for example annotation information or identification markings. For example, segmented areas of the anatomical structure can in each case be characterized as pertaining to a sub-structure of the anatomical structure. In particular, via a trained function the segmented areas can be labeled, i.e. characterized or annotated, especially simply and automatically, in particular also in a step containing the segmentation.

In accordance with one aspect of the proposed method the set of characteristic structure points can represent a center line of the anatomical structure. In particular, multiple center lines can also be represented if the anatomical structure comprises multiple sub-structures of interest. If the anatomical structure for example comprises at least one sub-region of the hand, a center line can be determined in each case for each finger, in particular also for the phalanges pertaining to a finger.

If the anatomical structure comprises part of the skeletal structure, a center line can be determined for each bone of the anatomical structure of interest. Furthermore, the provision of the two-dimensional model can include providing a specimen center line or multiple specimen center lines. The determination of the set of specimen points is then based on a mapping of the set of structure points to the specimen center axis or axes. In particular, the determination of the set of specimen points can in each case include determining a point at an identical relative position inside the specimen center line as a counterpart to a characteristic structure point on a center line in the model.

The center line of the anatomical structure can advantageously be determined on the basis of the segmented anatomical structure. For example, a distance transformation-based skeletonization algorithm (“DTF skeletonization”) can be applied to the segmented structure (see for example: Boskamp T et al. Geometrical and structural analysis of vessel systems in 3D medical image datasets. https://www.worldscientific.com/doi/pdf/10.1142/9789812701046_ 0001). For example, a method as per Lee T. C. et al (Lee T. C. et al, Building Skeleton Models via 3-D Medial Surface Axis Thinning Algorithms. https://doi.org/10.1006/cgip.1994.1042) or a similar method could be applied. It is also conceivable for example for a center axis to be determined on the basis of a machine-learning method with or without a prior segmentation. For example, a previously mentioned trained function based on an nnUnet architecture can be trained to output a center axis of the anatomical structure directly as output data. Accordingly, training of the trained function on the basis of training data and comparative data comprising training center lines of anatomical training structures is effected.

Mapping the characteristic structure points to the characteristic specimen points can advantageously be facilitated, in particular in the case of extended structures comprising multiple sub-structures, if annotation information is also captured during a segmentation and/or a skeletonization, for example an affiliation to a sub-structure of the anatomical structure. In this way, sub-structures of the anatomical structure can advantageously easily be assigned to sub-structures of the model.

The use of the center line in the proposed method is an advantageously simple implementation, since it can make use of a large number of established algorithms for extracting the center line. Furthermore, it represents an advantageous and, for an observer, easy-to-understand representation of the anatomical structure in the unfolded 2D image dataset. In particular, this may mean that the 2D unfolded image of the anatomical structure based on X-ray imaging corresponds to a coronal section through its center, if the imaging had been performed with the anatomical structure lying flat on an examination table.

In accordance with a further aspect of the proposed method, additional supporting structure points can also be determined in the first 3D image dataset, which are arranged orthogonally to the center axis in the first 3D image dataset or, if multiple center axes were determined, in each case are arranged orthogonally with a respective center axis and/or which interpolate a transition between sub-units, i.e. sub-structures, of the anatomical structure.

Further criteria, in particular also as a function of present circumstances, for the determination of the additional supporting structure points can be provided. For example, in this case a location of the fingers, a curvature of a finger or the type of finger can be taken into account. For example, when there is a relatively large curvature of a finger a different criterion for the arrangement of additional supporting structure points can be provided than with a lesser curvature of a finger. For example, for a thumb, which is frequently present in a different orientation from the remaining fingers, a different criterion can be provided than for the remaining fingers of the hand.

The supporting structure points as further input points for the deformation algorithm can enable a check to be made on how the deformed deformation surface runs through the anatomical structure or the sub-structures thereof. In this way it can for example be ensured that the deformed planar deformation surface runs like a coronal plane in the standard anatomical position through the anatomical structure, for example a respective bone. Additional supporting structure points in transitions between sub-structures can advantageously also help to standardize how the deformed deformation surface runs through the transition areas.

Supporting specimen points in the model can then accordingly also be determined as a counterpart to the supporting structure points and associated surface points in the deformation surface can be determined as further input points for the deformation algorithm. During the deformation of the deformation surface each supporting surface point is then moved to the corresponding associated supporting structure point.

Supporting specimen points can for example be designed in accordance with a respective specimen center axis and in particular can be arranged along a straight line orthogonally to the associated specimen center axis and inside the plane of the model. In particular, supporting specimen points can be assigned to each specimen center axis, but supporting specimen points can also be assigned to just one subset. In particular, this is dependent on the provision of the additional supporting structure points in the first 3D image dataset.

In accordance with a further aspect of the proposed method the determination of the set of specimen points can include aligning the model and the anatomical structure to one another and/or adjusting the model to the anatomical structure.

For an advantageous mapping of the characteristic structure points to the model a previous alignment of the model and of the anatomical structure can be provided in the 3D image dataset. The 2D model of the anatomical structure can for example be carried out using an alignment of particular landmarks or using a center axis orientation, e.g. by calculating a main component analysis of the center axes or center axis segments. For example, a model comprising one or more specimen center axes can be placed in the 3D coordinate system of the 3D image dataset so that it is aligned with the center axis or the center axes of the anatomical structure in the 3D image dataset, e.g. by calculating a least squares method.

Furthermore, it can be provided that for example an adjustment of a length and/or width of the model or sub-units of the model to the anatomical structure or sub-structures thereof takes place in order to achieve an improved mapping of the characteristic structure points to the model. For example, the length or lengths of the one or more specimen center axes can be adjusted to the length of the corresponding center axis or axes of the anatomical structure. This can advantageously take account of different proportions in comparison to the original template and avoid expansions or compressions, even in the case of sub-structures truncated physically or at the boundaries of the image volume.

In accordance with a further aspect of the proposed method the method can further comprise a provision of a third 3D image dataset comprising the anatomical structure, a localization of the anatomical structure in the third 3D image dataset, a determination of a partial image dataset on the basis of the third 3D image dataset and the localized anatomical structure, and a provision of the partial image dataset as a first 3D image dataset.

In particular, the third 3D image dataset can map a larger image section than the first or second 3D image dataset. In particular, the first 3D image dataset can be a sub-volume of the third 3D image dataset and can be based on measured data from the same acquisition procedure as the third 3D image dataset. For example, the third 3D image dataset can be a whole-body image dataset or an abdomen image dataset. Not only but in particular in the case of complex accidents, large-scale 3D image datasets are frequently generated, in order to obtain a quick overview of the overall effects on a patient's body. Based on this, anatomical structures of interest can then be identified or localized and a sub-volume comprising these structures of interest can be used for an unfolded representation and further diagnosis.

A localization of the anatomical structure in the third 3D image dataset can for example be implemented via a trained function. For example, an approach can be applied as described in Zheng, Y et al. (3D Deep Learning for Efficient and Robust Landmark Detection in Volumetric Data. https://doi.org/10.1007/978-3-319-24553-9_69) or in Viola P. and Jones M. (Rapid object detection using a boosted cascade of simple features, https://doi.org/10.1109/CVPR.2001.990517.) or else in Ghesu et al. (An Artificial Agent for Anatomical Landmark Detection in Medical Images. https://doi.org/10.1007/978-3-319-46726-9_27).

Based on the localized anatomical structure in the third 3D image dataset a sub-volume of the 3D image dataset, i.e. the partial image dataset, can be determined, which in particular comprises the anatomical structure, wherein image areas of the third 3D image dataset which are of less interest are no longer included. Based on a partial image dataset more efficient and more robust further processing can advantageously be effected in the proposed method.

In accordance with a further aspect of the proposed method a further similar anatomical structure can be at least partially included by the first three-dimensional image dataset, wherein the determination of the set of characteristic structure points can then include a separation of the anatomical structure and of the further similar anatomical structure.

In accordance with a further aspect of the proposed method a machine-learning method can be employed in at least one step of the method.

A machine-learning method can for example as already described above be employed in the step of the determination of the set of structure points and/or in the step of segmentation and/or in the step of localization.

One of these steps can advantageously be based on machine learning, in particular an application of a trained function to the first or third three-dimensional image dataset as input data. In this case input data of the trained function can be based on the image dataset, in particular can comprise the image dataset. Further, the set of structure points, the segmented anatomical structure, or the localization of the anatomical structure or of the partial image dataset can be provided as output data of the trained function or in a following step can also serve as inputs for a further trained function.

The trained function can be trained by a machine-learning method. In particular, the trained function can be a neural network, in particular a convolutional neural network (CNN) or a network comprising a convolutional layer. The trained function maps input data to output data. Here, the output data can still depend on one or more parameters of the trained function. The one or more parameters of the trained function can be determined and/or adjusted by training. The determination and/or adjustment of the one or more parameters of the trained function can in particular be based on a pair consisting of training input data and associated training output data, in particular comparative output data, wherein the trained function is applied for the provision of the set of structure points, the segmented anatomical structure, the localization of the anatomical structure or of the partial image dataset. In particular, the determination and/or adjustment can be based on a comparison of the training mapping data and the training output data, in particular the comparative output data. If for example the trained function serves for a segmentation, 3D training image datasets comprising the anatomical structure and comparative image datasets comprising the segmented anatomical structure can be provided for the training, and can then be compared to the output of the trained function. In general a trainable function, i.e. a function with one or more parameters which have not yet been adjusted, is referred to as a trained function.

Other terms for trained functions are trained mapping rule, mapping rule with trained parameters, function with trained parameters, algorithm based on artificial intelligence, and machine-learning algorithm. An example of a trained function is an artificial neural network, wherein the edge weights of the artificial neural network correspond to the parameters of the trained function. Instead of the term “neural network” the term “neural net” can also be used. In particular, a trained function can also be a deep (artificial) neural network. Another example of a trained function is a “support vector machine”, and furthermore other machine-learning algorithms can also in particular be employed as a trained function.

The trained function, in particular the neural network, advantageously has an input layer and an output layer. In this case the input layer of the trained function can be designed to receive the input data. Further, the output layer can be designed to provide mapping data, in particular output data. Further, the input layer and/or the output layer can each comprise multiple channels, in particular neurons. At least one parameter of the trained function can advantageously be adjusted on the basis of a comparison between a training dataset and a comparative dataset, for example comprising a segmented structure or a localized anatomical structure or a set of determined structure points.

Examples of possible concrete architectures which can be employed in the context of the proposed method in the sub-steps of the method are in this case already mentioned above.

In accordance with a further aspect of the proposed method the method can comprise dividing the anatomical structure into a plurality of sub-units, wherein at least the steps of the calculation of the first deformed deformation surface and of the calculation of the set of voxel positions is performed separately for each sub-unit, and wherein the calculation of the two-dimensional unfolded image dataset is based on the voxel positions of the plurality of sub-units.

Strong offsets or bends between characteristic structure points, for example of center lines, can lead to complex deformations of the deformation surface. This can cause the deformed deformation surface to form loops or overlaps, which can lead to artifacts, in which for example structures or deformations are represented twice. To avoid this, it may be advantageous to divide the anatomical structure into sub-units and perform a separate unfolding for each sub-unit. A composition into a common unfolded 2D image dataset is then performed. For example, the fingers of a hand can in each case represent the sub-units of the anatomical structure.

In accordance with a further aspect of the proposed method at least one second deformed deformation surface can be determined, in that the first deformed deformation surface is deformed along its location-dependent normal vectors. Based on the second deformed deformation surface and the first or second 3D image dataset, a second unfolded 2D image dataset can then be determined. The calculation of the second unfolded 2D image dataset can be effected analogously to the calculation of the first unfolded 2D image dataset. In particular, more than two deformed deformation surfaces can then be determined on the basis of a location-dependent deformation of the first deformed deformation surface along their location-dependent normal vectors, on the basis of which more than two unfolded 2D image datasets can accordingly be determined.

By determining at least one second deformed deformation surface and a corresponding unfolded 2D image dataset at least one second slice of the anatomical structure can be mapped in an unfolded representation. When calculating a plurality of deformed deformation surfaces a so-called 3D stack of the anatomical structure can advantageously be provided in an unfolded representation.

When for example using characteristic structure points, which each represent the center axis or center axes of the anatomical structure, the unfolded 2D image dataset corresponds to a coronal section through the center, if the imaging had taken place with the anatomical structure lying flat on the table. By determining a second or third deformed deformation surface, parts of the anatomical structure can be visualized anteriorly or posteriorly.

For example, a deformed surface lattice can be further deformed by shifting all lattice nodes/vertices gradually in the direction of their normal vectors.

An unfolded image dataset can then be created anew, by calculating the original image values of the first or second 3D image dataset in the same manner as described previously.

A stack of unfolded 2D image datasets can advantageously be provided on the basis thereof, which visualizes the anatomical structure in different slices. For example, 3D representations (e.g. “cinematic rendering”) based on the stack can also be generated at an unfolded 2D image dataset.

In accordance with a further aspect of the proposed method the provided second 3D image dataset can map the same image section as the first 3D image dataset, but can have a different image resolution than the first 3D image dataset and/or can comprise spectral image information.

As already described, a provided second 3D image dataset can in this case be an image dataset which is based on measured data from the same acquisition procedure as the first 3D image dataset and thus the location and position of the anatomical structure in the second 3D image dataset is present unchanged relative to the first 3D image dataset. For example, the first 3D image dataset or a third 3D image dataset on which a first 3D image dataset is based may have a coarser image resolution than the second 3D image dataset. For example, for the first or third 3D image dataset an image resolution can be reconstructed which is coarser than possible physically and via the measured data, in order to enable a time-efficient reconstruction and processing of the data in the proposed method. However, for the calculation of a higher-resolution unfolded image dataset a 3D image dataset can still be used, which is reconstructed with a higher image resolution. Such high-resolution unfolded image datasets can advantageously help to better visualize small structures and possible violations.

Likewise spectral measured data can be used to provide a 3D image dataset which comprises spectral information. For example, a 3D image dataset based on a material breakdown can be provided. Here too, an analogously unfolded 2D image dataset can be provided, which accordingly represents a two-dimensional mapping of the material breakdown maps, for example an iodine map or a bone marrow map. Such image data can then for example be shown superimposed in the 2D or 3D visualizations of the unfolded image data, in order to highlight vascular anomalies or microtraumatic bone injuries.

In accordance with a further aspect of the proposed method the method can also include the following:
the first or second unfolded 2D image dataset is displayed in a user interface,
a 2D position in the first or second two-dimensional unfolded image dataset is determined on the basis of a manual user input,
a 3D position in the first, second or third three-dimensional image dataset is determined on the basis of the 2D position and a result of the deformation algorithm, and
an image section of the first, second or third 3D image dataset and/or a cross-sectional representation thereof comprising the determined 3D position and/or a marking representing the 3D position in the first, second or third 3D image dataset and/or in the cross-sectional representation is displayed on the user interface.

The proposed method can also include the following:
the first, second or third 3D image dataset and/or at least one cross-sectional representation thereof is displayed via a user interface,
a 3D position in the first, second or third 3D image dataset and/or the cross-sectional representation is determined on the basis of a manual user specification,
a 2D position in the first or second unfolded 2D image dataset is determined on the basis of the 3D position and a result of the deformation algorithm,
an image section of the first or second unfolded 2D image dataset comprising the determined 2D position and/or a marking representing the 2D position in the first or second or the further unfolded 2D image dataset is displayed on the user interface.

Since advantageously a bidirectional assignment exists between 3D positions in the first or second 3D image dataset and 2D positions in the unfolded 2D image dataset, the corresponding position in the original 3D image dataset can be determined if a 2D position is selected in the unfolded image (“2D 3D navigation”) and vice versa (“3D 2D navigation”). This can also be extended to positions in the adjoining sections of a browsable stack, described above, of multiple unfolded 2D image datasets. User-controlled navigation between the 3D and 2D image datasets and a corresponding representation via suitable user interfaces, for example in the form of a display unit, for instance a monitor or display, and a keyboard and/or mouse, or else comprising a touchscreen, can advantageously be enabled, which permit a manual user input and the display of the image data.

A displayed image section can for example be selected to be centered around the marked position. The displayed image section in the case of a 3D image dataset can also be displayed in the form of multiple, in particular three, orthogonal sectional images, which for example are centered around the marked position. A marking can comprise a colored or other highlighting of the marked position, for example in the form of a marker which in the display is for example displayed superimposed on the image data.

In a further aspect, one or more example embodiments of the present invention additionally relate to a provision unit, which is designed to execute a proposed method and the variants thereof.

The advantages of the proposed provision unit substantially correspond to the advantages of the proposed method for providing at least one two-dimensional unfolded image dataset. Features, advantages or alternative forms of embodiment mentioned here can likewise be transferred to the other claimed subject matters and vice versa.

The provision unit can comprise a computing unit, a storage unit and/or an interface. The provision unit, in particular the components of the provision unit, can be designed to execute the individual steps of the proposed method for providing at least one first two-dimensional unfolded image dataset and the above-described variants. The interface can be designed to provide the first, second and/or third three-dimensional image dataset. It can additionally be designed to provide the two-dimensional model of the anatomical structure and to provide the first and/or second two-dimensional unfolded image dataset. The computing unit and/or the storage unit can be for the determination of the set of characteristic structure points, for the determination of the set of characteristic specimen points, for the determination of the deformation surface, for the calculation of the first and/or second deformed deformation surface, for the calculation of the voxel positions and for the calculation of the two-dimensional unfolded image dataset. Likewise, the computing unit and/or the storage unit can be designed to localize the anatomical structure in the third, three-dimensional image dataset and to determine the partial image dataset. The provision unit can itself also be designed to calculate, i.e. in particular to reconstruct, image datasets on the basis of provided measured data.

The provision unit can additionally comprise a user interface in the form of a display unit, which is designed to display the first, second and/or third three-dimensional image dataset and/or cross-sectional representations thereof. This can be implemented in the form of a monitor, a touchscreen or another suitable display device. The provision unit can additionally comprise a user interface in the form of an input unit, which is designed to enable a manual user input. This can for example be enabled via a keyboard and mouse with a corresponding input option or via another suitable input unit. Likewise, the input unit can be integrated into the display unit, for example in the form of a capacitive and/or resistive input display. In this case the input unit is additionally able, by a user input, to control the proposed method for providing a two-dimensional unfolded image dataset.

Furthermore, in a further aspect, one or more example embodiments of the present invention relate to a system comprising a medical imaging device and a provision unit for providing a two-dimensional unfolded image dataset in accordance with one of the above-described variants.

The medical imaging device can be designed to acquire and/or provide the first, second and/or third three-dimensional image dataset.

The medical imaging device can preferably be designed as an X-ray imaging system, in particular a CT system, with a radiation source and a radiation detector. It can also for example be designed as a magnetic resonance tomography system (MRT) and/or a medical X-ray device and/or a positron emission tomography system (PET). Further, the medical imaging device can be designed to acquire the first, second and/or third three-dimensional image dataset of an object under examination including the region of interest, i.e. in particular including the anatomical structure, and to provide it for the provision unit. Alternatively, the medical imaging device can be designed to acquire first, second and/or third image data, in particular first, second and/or third projection mappings, of the object under examination including the anatomical structure. In this case the medical imaging device can further be designed to provide the image data for the provision unit, wherein the provision unit is designed to reconstruct the first, second and/or third 3D image dataset accordingly from the first, second or third image data and to provide it for the further method.

In particular, the medical imaging device can be designed as an X-ray imaging system, in particular a CT device, comprising a photon-counting X-ray detector. By direct-conversion, photon-counting X-ray detectors, for example on the basis of a semiconductor material such as CdTe or CdZnTe or similar, small pixel sizes and thus a high spatial resolution can be achieved in the detector. Measured data can advantageously be provided which has a high spatial resolution, which can be transferred into a high image resolution of the reconstructed image datasets. The image resolution can in this case be adjustable by a possible prior merging of measured data and/or the reconstruction parameters used. This can advantageously favor improved diagnostics on the basis of the image datasets.

Furthermore, the photon-counting X-ray detector can be designed to be energy-resolving. In this way, spectral information, for example with regard to a material distribution, can also be provided. Additional information can advantageously be provided, which can likewise favor improved diagnostics on the basis of the image datasets.

The advantages of the proposed medical imaging device substantially correspond to the advantages of the proposed method for providing the two-dimensional unfolded image dataset and/or the proposed provision unit. Features, advantages or alternative forms of embodiment mentioned here can likewise also be transferred to the other claimed subject matters and vice versa.

In a further aspect, one or more example embodiments of the present invention additionally relate to a non-transitory computer program product with a computer program which can be loaded into a memory of a provision unit for the provision of at least one two-dimensional unfolded image dataset in accordance with one of the above-described variants, with program sections in order to execute all steps of the method for providing at least one two-dimensional unfolded image dataset and the above-described variants thereof if the program sections are executed by the provision unit. The computer program product can in particular be stored on a non-transitory computer-readable storage medium.

The advantages of the proposed computer program product or computer-readable storage medium substantially correspond to the advantages of the proposed method for providing a two-dimensional unfolded image dataset. Features, advantages or alternative forms of embodiment mentioned here can likewise also be transferred to the other claimed subject matters and vice versa.

The computer program product can for example comprise software with a source code which still has to be compiled and linked or only has to be interpreted, or an executable software code which for execution still has to be loaded into the provision unit. Thanks to the computer program product the method for providing a two-dimensional unfolded image dataset via a provision unit can be executed quickly, identically repeatably and robustly. The computer program product is configured so that via the provision unit it can execute the inventive method steps.

The computer program product is for example stored on a computer-readable storage medium or on a network or server, from where it can be loaded into the processor of a provision unit, it being possible for said processor to be directly connected to the provision unit or to be designed as part of the provision unit. Furthermore, control information of the computer program product can be stored on an electrically readable data carrier. The control information of the electronically readable data carrier can be designed such that it performs an inventive method when the data carrier is used in a provision unit. Examples of electronically readable data carriers are a DVD, a magnetic tape or a USB stick, on which electronically readable control information, in particular software, is stored. If this control information is read from the data carrier and stored in a provision unit, all inventive forms of embodiment of the method described above can be performed.

A largely software-based implementation has the advantage that provision units used hitherto can be easily retrofitted by a software update in order to work in the inventive manner. Besides the computer program, such a computer program product can include additional components, for example documentation and/or additional components, as well as hardware components, for example hardware keys (dongles, etc.) for the use of the software.

### BRIEF DESCRIPTION OF THE DRAWINGS

Exemplary embodiments of the present invention are illustrated in the drawings and are described in greater detail below. The same reference characters are used in different figures for identical features. In the drawings:

FIG. 1 shows a schematic flow diagram of the proposed method for providing at least one two-dimensional unfolded image dataset of an anatomical structure,

FIG. 2 shows a schematic flow diagram of the step of the determination of the set of characteristic structure points in accordance with one aspect of the proposed method,

FIG. 3 shows a schematic flow diagram of the step of the determination of the set of characteristic specimen points in accordance with one aspect of the proposed method,

FIG. 4 shows a schematic flow diagram for the provision of a partial image dataset of a 3D image dataset as a first 3D image dataset in accordance with one aspect of the proposed method,

FIG. 5 shows a schematic flow diagram of the steps of the calculation of a deformed deformation surface, of the calculation of a set of voxel positions and of the calculation of the unfolded 2D image dataset on the basis of sub-units of the anatomical structure in accordance with one aspect of the proposed method,

FIG. 6 shows a schematic flow diagram for the determination of a second unfolded 2D image dataset on the basis of the first deformed deformation surface in accordance with one aspect of the proposed method,

FIG. 7 shows a schematic flow diagram in accordance with one aspect of the proposed method, wherein 2D and associated 3D positions are determined and displayed in the unfolded 2D image dataset or 3D image dataset,

FIG. 8 shows a further schematic representation of an operational sequence of the proposed method in accordance with one embodiment variant,

FIG. 9 shows a schematic representation of a determined set of characteristic structure points and associated characteristic specimen points using the example of extracted center lines of a hand as an anatomical structure,

FIG. 10 shows a simplified, schematic illustration for the determination of a second deformed deformation surface on the basis of a first deformed deformation surface in accordance with one aspect of the proposed method,

FIG. 11 shows a schematic representation of a provision unit, which is designed to execute a proposed method and aspects thereof,

FIG. 12 shows a schematic representation of a system having a medical imaging device and a provision unit.

### DETAILED DESCRIPTION

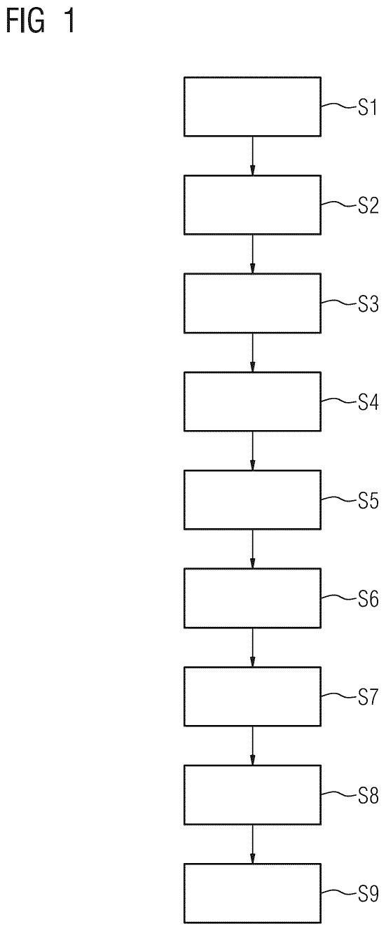

FIG. 1 shows a schematic flow diagram of the proposed computer-implemented method for providing at least one first unfolded 2D image dataset EBDS1 of an anatomical structure AS.

The method can start with the provision S1 of a first 3D image dataset BDS1, which comprises the anatomical structure. The anatomical structure can in particular be an anatomical structure of a human and/or animal patient or can be comprised by an examination phantom. For example, the anatomical structure can comprise at least one sub-region of the hand, of the arm or of the foot.

In a further step S2 a set of characteristic structure points SP of the anatomical structure based on the first three-dimensional image dataset BDS1 is determined. The set of characteristic structure points SP can particularly advantageously represent a center line of the anatomical structure AS or multiple center lines of the anatomical structure AS, if this comprises multiple sub-units. For example, the set of characteristic structure points SP can be located on the center lines of the fingers, in particular the phalanges pertaining to the fingers. The determination of the set of characteristic structure points SP can precede a segmentation S21 of the anatomical structure AS in the 3D image dataset. A segmentation S21 via a machine-learning method can particularly advantageously be implemented, i.e. with a trained function. Based on a segmented anatomical structure, for example via a skeletonization algorithm, for example a DTF skeletonization, the center line or lines of the anatomical structure can particularly advantageously be extracted.

Furthermore, it can happen that a further, similar anatomical structure is included by the first three-dimensional image dataset EBDS1 at least partially. For example, at least one second hand can be mapped at least partially in the first 3D image dataset. In this case the method can include determining the set of characteristic structure points and separating the anatomical structure and the further anatomical structure, in particular also after a segmentation.

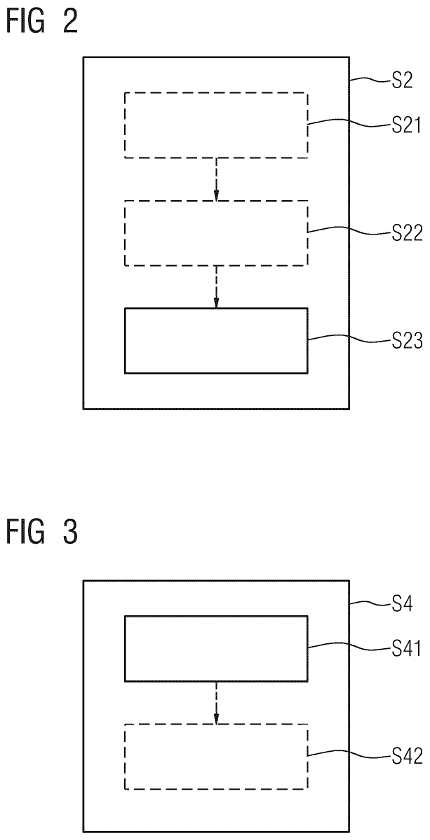

As also shown in FIG. 2, the step of the determination of the set of characteristic structure points SP can for example include performing a segmentation S21 of the anatomical structure in the first 3D image dataset BDS1, carrying out a separation S22 of the anatomical structure AS from a further similar anatomical structure, if present, and then for example determining a set of characteristic structure points SP which represents S23 a center line of the anatomical structure. In other embodiment variants the step S2 can also be configured differently. For example, it can also be provided that a center line is extracted directly from the first 3D image dataset BDS1, for example via a machine-learning method.

A separation S22 can in this case be achieved using localization and segmentation of the anatomical structures and a mask generated based thereon to mask out the further anatomical structure, in particular also after a determination of characteristic structure points. Also, directly from the segmentation in particular via a trained function, an assignment or identification marking of the segmented structures can follow as pertaining to the one or other anatomical structure, on the basis of which a separation is enabled.

In a further step S3 a two-dimensional model of the anatomical structure AS is then provided. The model can also be referred to as a “template”. The model can represent a standardized representation of the anatomical structure AS. If for example the method relates to the hand or at least a sub-region of the hand, the 2D model can map the anatomy of a hand, in particular the arrangement of the bones of the hand, in a standardized form. The model can also map the anatomy of the anatomical structure AS in a reduced form. If for example the characteristic structure points SP include a center axis of the anatomical structure, the model can likewise include a provided specimen center axis of an anatomical specimen structure. In particular, the model can include further data over and above a straightforward mapping of the anatomy, for example annotation information such as for instance identification markings of anatomical sub-structures or anatomical landmarks. This can in particular facilitate a subsequent mapping of the characteristic structure points SP to the model.

In a next step S4 a set of characteristic specimen points MP is determined by mapping the set of structure points SP to the model. In this case a counterpart to a characteristic structure point SP at an identical relative position inside the anatomical structure can in particular in each case be determined in the model. If the set of characteristic structure points SP represents one or more center lines of the anatomical structure AS and if the provision S3 of the two-dimensional model includes providing one or more specimen center lines, the determination S4 of the set of specimen points MP is based in particular on a mapping of the set of characteristic structure points SP to the specimen center line(s).

In accordance with one aspect of the proposed method, as indicated in FIG. 3, the determination S4 of the set of specimen points MP can include an alignment S41 of the model and of the anatomical structure AS in the first three-dimensional image dataset BDS1 to one another, for example on the basis of an alignment of determined landmarks or on the basis of a center axis orientation. An adjustment S42 of the model to the anatomical structure AS can also be included, for example a length and/or width adjustment of the model or sub-units of the model to the anatomical structure AS or sub-structures thereof.

In a further step S5 a deformation surface in a plane with the set of specimen points MP is determined, wherein each specimen point of the set of specimen points MP is represented by a surface point in the deformation surface. The deformation surface can in particular have a smooth geometry and in advantageous embodiments can include a (surface) lattice, in particular a regular lattice. If the deformation surface is provided as a lattice, a surface point can in each case correspond to the lattice node of the lattice, said lattice node being nearest to a specimen point. Each of the surface points can also in each case correspond to a lattice node point of the surface lattice, which in the initial undeformed deformation surface is nearest to the specimen point MP from the set of specimen points MP, which is represented by the corresponding surface point. There can also be other embodiments.

In a further step S6 a first deformed deformation surface DF1 is calculated by applying a deformation algorithm to the initial undeformed deformation surface, the set of structure points SP and the set of surface points, wherein each surface point from the set of surface points is moved to the corresponding structure point SP from the set of structure points SP. The deformation algorithm can for example be based on an “As-Rigid-As-Possible” algorithm, wherein the structure points SP or surface points serve as “constraint points” for the deformation of the initial undeformed deformation surface.

In the further steps S7 and S8 the unfolded 2D image dataset EBDS1 is then generated on the basis of the first deformed deformation surface and the first 3D image dataset BDS1 or a provided second 3D image dataset. In step S7 a set of voxel positions in relation to the first three-dimensional image dataset BDS1 or in relation to the provided second three-dimensional image dataset and based on the first deformed deformation surface DF1 is determined. The two-dimensional unfolded image EBDS1, in particular the image values of the pixels of the unfolded 2D image dataset EBDS1, can then be scanned, i.e. calculated, by extracting voxel values from the voxel positions in the first or second 3D image dataset (step S8). As a result, a bidirectional assignment between 3D positions in the first or second 3D image dataset and 2D positions in the unfolded 2D image dataset can additionally be created. The determination of the voxel positions or of the image values extracted therefrom can include an interpolation, since a deformed deformation surface does not necessarily run through the voxel centers of a 3D image dataset, these normally being arranged in a fixed grid.

The provided second 3D image dataset can in this case in particular represent the same imaging area as the first 3D image dataset BDS1. However, the second 3D image dataset can differ from the first 3D image dataset BDS1 for example with regard to a reconstructed image resolution or an information content, for example relating to spectral image information. The second 3D image dataset can in particular be based on measured data from the same acquisition procedure as the first 3D image dataset. If the second 3D image dataset differs from the first 3D image dataset BDS1 with regard to an image resolution, it can be provided for a calculation of the voxel positions and of the unfolded image dataset EBDS1 that an edge length of the polygons of a planar surface lattice representing the deformation surface and/or a number of the points scanned per polygon is adjusted in order to adjust the resolution of the resulting unfolded 2D image dataset EBDS1 to the resolution of the second 3D image dataset.

In a further step S9 the two-dimensional unfolded image dataset EBDS1 is then provided.

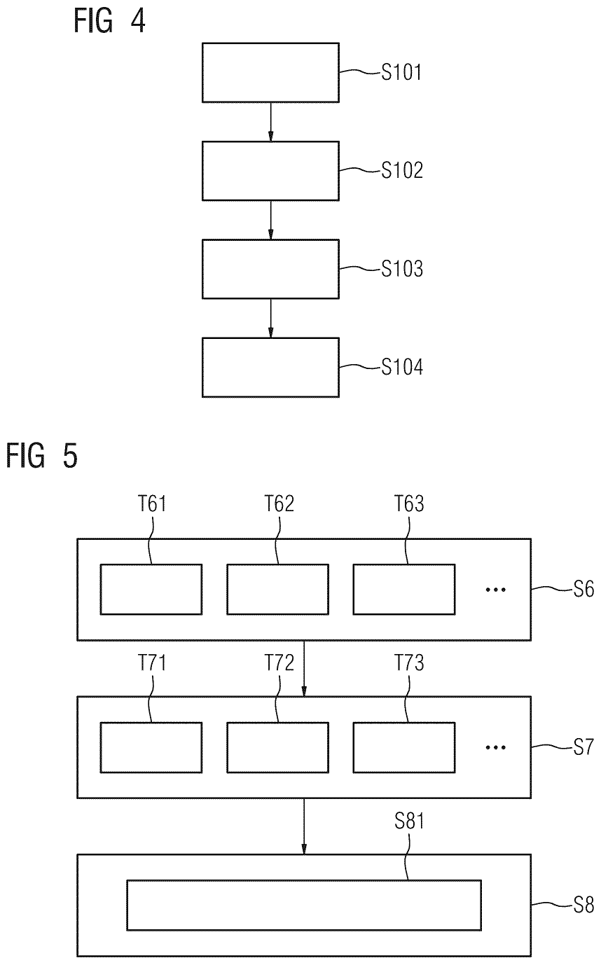

FIG. 4 schematically further shows a method variant, wherein the provision of the first 3D image dataset EBDS1 is based on a previously determined partial image dataset TB from a third 3D image dataset.

This can include the provision S101 of a third three-dimensional image dataset BDS3 comprising the anatomical structure AS, the localization S102 of the anatomical structure in the third three-dimensional image dataset BDS3, the determination S103 of a partial image dataset TBS based on the third three-dimensional image dataset BDS3 and the localized anatomical structure, and the provision S104 of the partial image dataset TBS as a first three-dimensional image dataset BDS1.

In particular, the third 3D image dataset BDS3 can map a larger image section than the first or second 3D image dataset. In particular, the first 3D image dataset BDS1 can be a sub-volume of the third 3D image dataset and can be based on measured data from the same acquisition procedure as the third 3D image dataset BDS3.

For example, a localization S103 of the anatomical structure AS in the third 3D image dataset BDS3 can be based on the application of a trained function. For example, a sub-volume, i.e. a partial image dataset TBS, can be defined around the localized anatomical structure AS, and includes the anatomical structure, wherein the latter is then provided to the proposed method as a first 3D image dataset BDS1.

FIG. 5 shows a further method variant, wherein the anatomical structure AS is divided into a plurality of sub-units and at least the steps of the calculation S6 of the first deformed deformation surface DF1 and of the calculation S7 of the set of voxel positions for each sub-unit are performed separately, and wherein the calculation of the two-dimensional unfolded image dataset S8 is based on the voxel positions of the plurality of sub-units. This means that for each sub-unit at least one step of the calculation T61, T62, T63, . . . of the first deformed deformation surface DF1 and one step of the calculation T72, T72, T73 . . . of the set of voxel positions is performed separately, wherein for the calculation S8 of the unfolded 2D image dataset the partial unfoldings are combined S81 to form a common unfolded image dataset.

In particular, if the anatomical structure AS includes a hand, then during the division into a plurality of sub-units a respective sub-unit can include a finger, in particular all phalanges pertaining to a finger.

In this case for the partial unfoldings supporting structure points can also be used as constraint points for the deformation, these being assigned to adjacent sub-units, in order to prevent mismatched unfoldings in overlapping image areas.

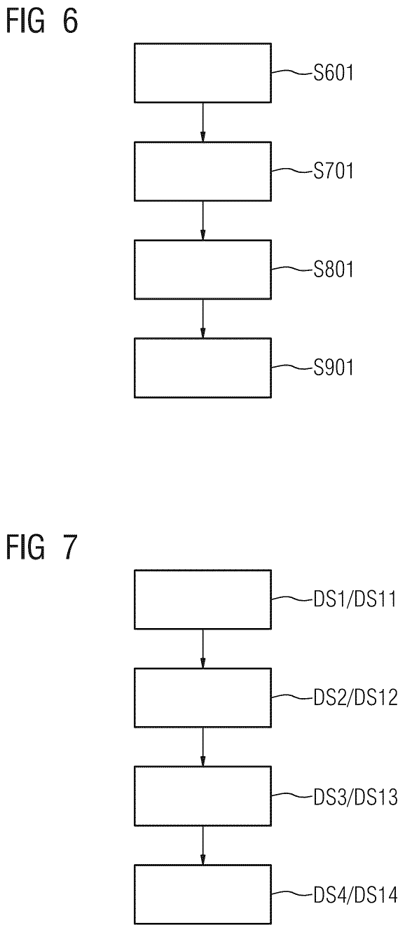

FIG. 6 further shows a schematic operational sequence of an aspect of the proposed method, wherein a second deformation surface and a second unfolded 2D image dataset is determined. To this end, in step S601 at least one second deformed deformation surface DF2 can be determined, in that the first deformed deformation surface DF1 is deformed along its location-dependent normal vectors. Based on the second deformation surface DF2 and the first or second 3D image dataset BDS1 a second unfolded 2D image dataset can be determined. This is also illustrated in FIG. 10 in a simplified representation and in a sectional view perpendicular to the deformation surface. For example, the result of the proposed method is a first deformed deformation surface DF1, which is based on a center line extraction of the anatomical structure AS as characteristic structure points SP, which runs through a center slice of the anatomical structure AS. By determining a second or third deformed deformation surface DF2 based on a location-dependent deformation of the first deformed deformation surface DF1 along its location-dependent normal vectors, slices of the anatomical structure can also be visualized anteriorly or posteriorly. Based on the second deformation surface DF2, analogously to the above description, a second unfolded 2D image dataset can be generated in the steps of the calculation S701 of a set of voxel positions in relation to the first three-dimensional image dataset BDS1 or in relation to a provided second three-dimensional image dataset and on the basis of the second deformed deformation surface DF2, calculation S801 of a second unfolded 2D image dataset of the anatomical structure AS based on the first three-dimensional image dataset BDS1 or the second three-dimensional image dataset and the set of voxel positions, and can be provided in the step S901.

In particular, in such a calculation of a plurality of deformed deformation surfaces a so-called 3D stack of the anatomical structure AS can be provided in an unfolded representation.

FIG. 7 shows a schematic flow diagram of a method in accordance with a further aspect.

The method here further includes, in a step DS1, displaying the first or second (and where appropriate also every further) unfolded 2D image dataset EBDS1 in a user interface 41.

Furthermore, the method includes the step DS2, in which a 2D position in the first or second unfolded 2D image dataset EBDS1 is determined on the basis of a manual user input.

Furthermore, in a step DS3 a 3D position in the first, second or third 3D image dataset BDS1 is determined on the basis of the 2D position and a result of the deformation algorithm/the deformed deformation surface, and in a step DS4 an image section of the first, second or third 3D image dataset BDS1 and/or a cross-sectional representation thereof comprising the determined 3D position and/or a marking representing the 3D position in the first, second or third 3D image dataset BDS1 and/or in the cross-sectional representation is displayed DS4 on the user interface 41.

Since advantageously a bidirectional assignment exists between 3D positions in the first or second 3D image dataset and 2D positions in the unfolded 2D image dataset, the corresponding position in the original 3D image dataset can be determined if a 2D position in the unfolded image is selected (“2D 3D navigation”).

The same also applies the other way round (“3D 2D navigation”). This means the method can also alternatively or likewise comprise a step DS11 in which the first, second or third 3D image dataset BDS1 and/or at least one cross-sectional representation thereof is displayed via a user interface 41, in a step DS12 a 3D position in the first, second or third 3D image dataset BDS1 and/or the cross-sectional representation is determined on the basis of a manual user specification, in a further step DS13 a 2D position in the first or second unfolded 2D image dataset is determined on the basis of the 3D position and a result of the deformation algorithm/the deformed deformation surface, and then in a step DS14 an image section of the first or second unfolded 2D image dataset EBDS1 comprising the determined 2D position and/or a marking representing the 2D position in the first or second unfolded 2D image dataset EBDS1 is displayed on the user interface 41.

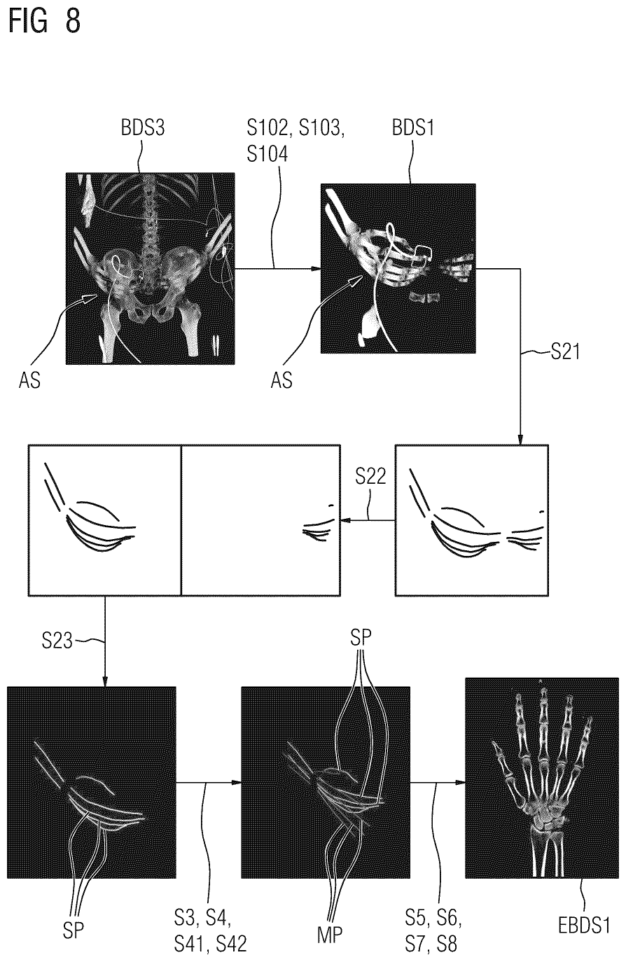

FIG. 8 illustrates a method sequence in accordance with a method variant. In this case the anatomical structure AS relates to the hand of a patient. In particular, a third 3D image dataset BDS3 is provided here, on the basis of which the anatomical structure AS is localized (S102) and a partial image dataset (S103) is determined, which in particular includes the localized anatomical structure. This partial image dataset is provided (S104) for the further steps as a first 3D image dataset BDS1.

Based on the first 3D image dataset BDS1 a segmentation S21 of the anatomical structure AS is effected, so that an image volume comprising the segmented structure is provided. In the case shown, a second similar anatomical structure, besides the anatomical structure of interest, is also at least partially included by the image volume, so that a separation S22 of the anatomical structures is performed.

Based on the separated and segmented anatomical structure a set of characteristic structure points SP is determined, which represent (S23) a center line of the segmented structure, here the center lines of the phalanges of the hand as well as of a part of the ulna and radius of the arm.

Furthermore, in the steps S3 and S4 (not shown) a model of the anatomical structure is provided and a set of specimen points is determined by mapping the characteristic structure points to the model. In this case an alignment S41 of the model and the anatomical structure AS to one another and/or an adjustment S42 of the model to the anatomical structure AS are in particular included. The next mapping in the method sequence in particular shows the aligned, characteristic structure points SP and the associated specimen points MP, which are used for a subsequent unfolding. In particular, in step S5 a deformation surface in a plane with the set of specimen points MP is determined, wherein each specimen point of the set of specimen points MP is represented by a surface point in the deformation surface, in step S6 a first, deformed deformation surface DF1 is calculated by applying a deformation algorithm to the deformation surface, the set of structure points SP and the set of surface points, in step S7 a set of voxel positions is calculated in relation to the first three-dimensional image dataset BDS1 and on the basis of the first deformed deformation surface DF1 and in a step S8 the first two-dimensional unfolded image dataset EBDS1 of the anatomical structure AS is calculated on the basis of the first three-dimensional image dataset BDS1 and the set of voxel positions, so that this can ultimately be provided.

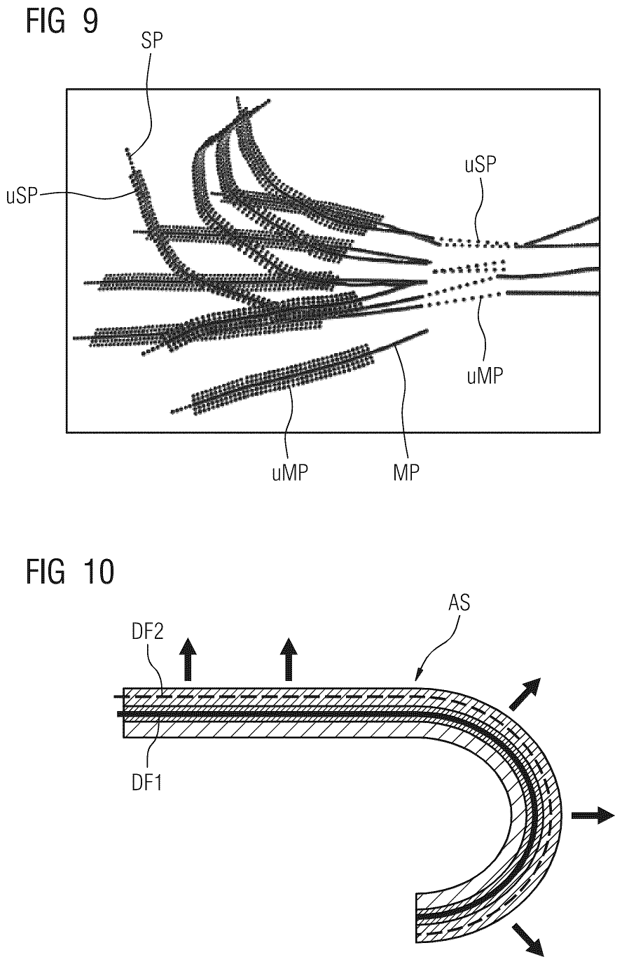

FIG. 9 further illustrates an aspect of the proposed method, wherein additional supporting structure points uSP are also determined, which are arranged orthogonally to the center line in the first 3D image dataset or, if multiple center lines have been determined, in each case orthogonally with a respective center line and/or which interpolate a transition between sub-structures of the anatomical structure AS.

The figure illustrates extracted characteristic structure points SP, which are arranged on the center lines of the fingers of a hand and in addition also on a part of the ulna and radius. In addition, supporting structure points uSP are determined for each center line, these being oriented to the extracted center lines and orthogonal thereto. Furthermore, supporting structure points uSP are also provided in a transition area between the phalanges and the ulna and radius in the area of the carpal bones, in which no center lines are present.

Further criteria, in particular also as a function of existing circumstances, can be provided for the determination of the additional supporting structure points.

For example, the supporting structure points uSP lie on lines orthogonal to the extracted center lines. For example, in the case of a smaller curvature of a finger, for example <=60°, wherein the curvature of a finger for example consists of an angle between the corresponding metacarpals of the hand and the associated distal phalanx, for instance the angle between the vectors determined from the first and last n center axis points, the supporting structure points uSP furthermore also lie orthogonally to the z-axis of the hand, which for instance can be determined via a PCA (Principal Component Analysis), wherein the z-axis is the shortest main axis. For example, in the case of a larger curvature of a finger, for example >60°, the supporting structure points uSP are furthermore based on a plane which was fitted onto the center axis points of the respective finger (i.e. the plane for which e.g. the sum of the squares of the distances of the points from the plane is minimal) and for example for the thumb lie inside this plane, i.e. on lines orthogonal to the normals of the plane and for the other fingers on lines in the direction of the normals of the plane (i.e. perpendicular on the plane).

Accordingly, supporting specimen points uMP in the model aligned with the anatomical structure AS in the form of pattern centerlines are also determined as a counterpart to the supporting structure points uSP, and associated surface points in the deformation surface are determined as further input points for the deformation algorithm. The supporting specimen points uMP are likewise oriented on the center lines of the model and are arranged in a plane with the model and in the transition area between the phalanges and the bones of the arm.

The supporting structure points uSP and corresponding supporting specimen points or surface points as further input points for the deformation algorithm can enable a check to be made on how the deformed deformation surface DF1 runs through the anatomical structure AS or the sub-structures thereof. In this way it can for example be ensured that the deformed deformation surface DF1 runs approximately like a coronal plane in the standard anatomical position through the anatomical structure AS, for example a respective bone. In addition, additional supporting structure points in transitions between sub-structures can advantageously help to standardize how the deformed deformation surface runs through the transition areas.

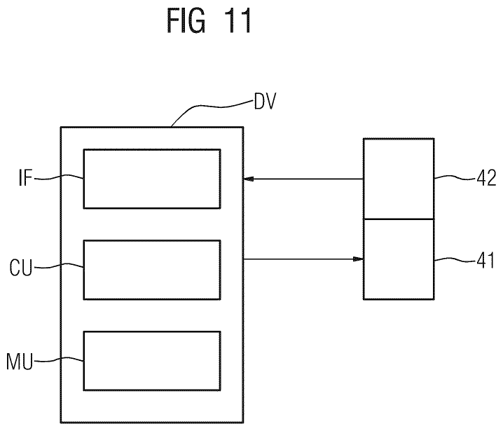

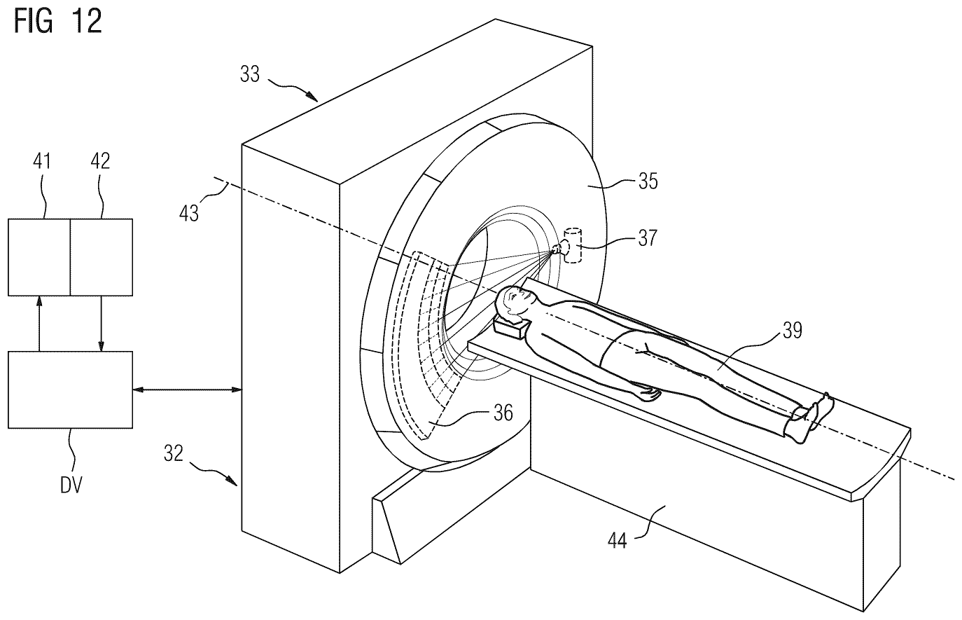

FIG. 11 shows a schematic representation of a provision unit DV, which is designed for the execution of a proposed method and aspects thereof. The provision unit DV can include an interface IF, a computing unit CU and a storage unit MU.

The components of the provision unit DV can be connected to one another in order to enable an efficient exchange of data. The interface IF can be directly connected to the computing unit CU, which in turn can be connected to the storage unit MU. This arrangement can enable a flow of information from the interface IF to the storage unit MU via the computing unit CU.

The provision unit DV can be designed for data processing and storage. The interface IF can in this case serve as an interface for input/output operations, the computing unit CU can perform calculation tasks, and the storage unit MU can store data or results.

The interface can in particular be designed to provide a first, second or third 3D image dataset and/or a model. The interface can be designed to provide a first or second unfolded 2D image dataset. The computing unit CU can be designed to execute the steps of the determination and calculation of the proposed method and corresponding steps of its embodiment variants.

The storage unit MU can be designed to store a first, second or third 3D image dataset and/or a model and/or a first or second unfolded 2D image dataset. The storage unit MU can use various storage technologies to enable efficient management and fast access to the stored data.

The provision unit DV can have an input unit 42, for example a keyboard, and a display unit 41, for example a monitor and/or display. The input unit 42 can be integrated into the display unit 41, for example in the case of a capacitive and/or resistive input display. In this case control of the provision unit DV can be enabled by an input by a medical operative at the input unit 42. For this, the input unit 42 can for example send a signal to the provision unit DV. The display unit 41 can advantageously be designed to display 2D and 3D image datasets and information and representations derived therefrom, for example cross-sectional representations. For this, the provision unit DV can send a signal to the display unit 41. Furthermore, via the input unit 42 and where appropriate the display unit 41 a manual input by a medical operative can be enabled, for example to determine a 2D or 3D position in a 2D or 3D image dataset in accordance with one aspect of the proposed method, which can be further processed via the provision unit DV.

FIG. 12 shows a schematic representation of a system comprising a medical imaging device 32 and a provision unit DV.

In this example, the medical imaging device 32 is in particular a CT device comprising a radiation source, here in particular an X-ray source 37, a radiation detector, here in particular an X-ray detector 36, and a provision unit DV. In this case the X-ray source 37 and the X-ray detector 36 can be arranged in juxtaposition to one another. The X-ray source 37 can be designed to illuminate the X-ray detector 36 with X-rays along an X-ray direction of incidence. The X-ray detector 1 can be an indirect-conversion X-ray detector, for example a scintillation detector or else a direct-conversion X-ray detector. In particular, a direct-conversion X-ray detector can be designed as a photon-counting X-ray detector, for example having CdTe, CdZnTe, CdTeSe, CdZnTeSe or CdMnTe or else another semiconductor material as a detector material. A very high image resolution achievable with a photon-counting X-ray detector and/or the access to spectral information can advantageously also be used in the context of the proposed method.

The CT device 32 can moreover include a gantry 33 with a rotor 35. The X-ray source 37 and the X-ray detector 36 can be arranged on the rotor 35 in a defined arrangement, in particular integrated into the rotor 35 or attached to the rotor 35. The rotor 35 can be rotatably mounted about an axis of rotation 43. The object under examination 39 to be mapped, here a human patient, can be mounted on the patient positioning apparatus 44 and can be moved along the axis of rotation 43 through the gantry 33. The processing unit DV can be used to control the CT device 32 and to calculate sectional images or volume images of the object under examination 39. An input device 42, for example a keyboard and/or mouse, and an output apparatus 41, for example a monitor and/or display, can be connected to the processing unit DV, in particular coupled using signal technology. The input facility 42 (or input device) can be integrated into the output apparatus 41, for example in the case of an input display, in particular a resistive and/or capacitive input display. The output apparatus 41 can be designed to display a graphical representation of the image dataset and or information and representations derived therefrom. Control of the provision unit DV and/or of the medical imaging device 32 can be enabled by an input by a medical operative at the input unit 42.

The schematic representations contained in the figures described do not depict any scale or proportions.

In conclusion it is once again noted that the methods described in detail above as well as the apparatuses shown relate solely to exemplary embodiments that can be modified by the person skilled in the art in a variety of ways, without departing from the scope of the present invention. Furthermore, the use of the indefinite article “a” or “an” does not rule out that the features in question may also be present multiple times. Likewise the terms “unit” and “element” do not rule out that the components in question consist of multiple interacting subcomponents which if appropriate may also be distributed spatially.

The expression “on the basis of” can in particular be understood, in the context of the present application, in the sense of the expression “using”. In particular, a wording according to which a first feature is generated (alternatively: determined, ascertained, etc.) on the basis of a second feature does not rule out that the first feature can be generated (alternatively: determined, ascertained, etc.) on the basis of a third feature.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (device) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for providing at least one two-dimensional unfolded image dataset of an anatomical structure, the computer-implemented method comprising:
provisioning a first three-dimensional image dataset, which includes the anatomical structure;
determining a set of characteristic structure points of the anatomical structure based on the first three-dimensional image dataset;
provisioning a two-dimensional model of the anatomical structure;
determining a set of characteristic specimen points by mapping the set of characteristic structure points to the two-dimensional model;
determining a deformation surface in a plane with the set of characteristic specimen points, wherein each specimen point of the set of characteristic specimen points is represented by a surface point, of a set of surface points, in the deformation surface;
calculating a first deformed deformation surface by applying a deformation algorithm to the deformation surface, the set of characteristic structure points and the set of surface points, wherein each surface point from the set of surface points is moved to a corresponding characteristic structure point from the set of characteristic structure points;
calculating a set of voxel positions relative to the first three-dimensional image dataset or relative to a second three-dimensional image dataset, and based on the first deformed deformation surface;
calculating a first two-dimensional unfolded image dataset of the anatomical structure based on the first three-dimensional image dataset or the second three-dimensional image dataset and the set of voxel positions; and
provisioning the first two-dimensional unfolded image dataset.

2. The computer-implemented method as claimed in claim 1, wherein determining of the set of characteristic structure points includes segmenting the anatomical structure to obtain a segmented anatomical structure, and wherein the set of characteristic structure points is determined based on the segmented anatomical structure.

3. The computer-implemented method as claimed in claim 1, wherein
the set of characteristic structure points represents a center line of the anatomical structure,
provisioning of the two-dimensional model includes providing a specimen center line, and
determining of the set of characteristic specimen points is based on a mapping of the set of characteristic structure points to the specimen center line.

4. The computer-implemented method as claimed in claim 3, further comprising:
determining a plurality of additional supporting structure points, the plurality of additional supporting structure points at least one of are arranged orthogonally to the center line of the anatomical structure or interpolate a transition between sub-structures of the anatomical structure.

5. The computer-implemented method as claimed in claim 1, wherein determining of the set of characteristic specimen points includes at least one of an alignment of the two-dimensional model and the anatomical structure to one another or an adjustment of the two-dimensional model to the anatomical structure.

6. The computer-implemented method as claimed in claim 1, wherein the deformation surface is designed as a surface lattice and each surface point corresponds to a lattice node point of the surface lattice which in an initial deformation surface lies nearest to a specimen point from the set of characteristic specimen points, which is represented by a corresponding surface point.

7. The computer-implemented method as claimed in claim 1, further comprising:
provisioning a third three-dimensional image dataset including the anatomical structure;
localizing the anatomical structure in the third three-dimensional image dataset;
determining a partial image dataset based on the third three-dimensional image dataset and the localized anatomical structure; and
provisioning the partial image dataset as the first three-dimensional image dataset.

8. The computer-implemented method as claimed in claim 1, wherein
a further similar anatomical structure is at least partially included in the first three-dimensional image dataset, and
the computer-implemented method includes separating the anatomical structure and the further similar anatomical structure prior to determining the set of characteristic structure points.

9. The computer-implemented method as claimed in claim 1, wherein a machine-learning method is employed in at least one step of the computer-implemented method.

10. The computer-implemented method as claimed in claim 1, wherein the anatomical structure is divided into a plurality of sub-units and at least calculating of the first deformed deformation surface and calculating of the set of voxel positions is performed separately for each sub-unit, and wherein calculating of the first two-dimensional unfolded image dataset is based on voxel positions of the plurality of sub-units.

11. The computer-implemented method as claimed in claim 1, further comprising:
determining at least one second deformed deformation surface by deforming the first deformed deformation surface along its location-dependent normal vectors; and
determining a second two-dimensional unfolded image dataset based on the at least one second deformed deformation surface and the first three-dimensional image dataset or the second three-dimensional image dataset.

12. The computer-implemented method as claimed in claim 1, wherein
the second three-dimensional image dataset maps a same imaging area as the first three-dimensional image dataset, and
the second three-dimensional image dataset at least one of has a different image resolution than the first three-dimensional image dataset or includes spectral image information.

13. The computer-implemented method as claimed in claim 1, wherein
the computer-implemented method further includes
displaying the first two-dimensional unfolded image dataset or a second two-dimensional unfolded image dataset on a user interface,
determining, based on a manual user input, a 2D position in the first two-dimensional unfolded image dataset or the second two-dimensional unfolded image dataset,
determining a 3D position in the first three-dimensional image dataset, the second three-dimensional image dataset or a third three-dimensional image dataset based on the 2D position and a result of the deformation algorithm, and
displaying at least one of
an image section of the first three-dimensional image dataset, the second three-dimensional image dataset or the third three-dimensional image dataset,
a cross-sectional representation including the 3D position, or
a marking representing the 3D position in at least one of the cross-sectional representation or the first three-dimensional image dataset, the second three-dimensional image dataset or the third three-dimensional image dataset; or
the computer-implemented method further includes
displaying, via a user interface, at least one of the first three-dimensional image dataset, the second three-dimensional image dataset, the third three-dimensional image dataset, or at least one cross-sectional representation thereof,
determining, based on a manual user specification, at least one of the at least one cross-sectional representation or a 3D position in the first three-dimensional image dataset, the second three-dimensional image dataset or the third three-dimensional image dataset,
determining a 2D position in the first two-dimensional unfolded image dataset or the second two-dimensional unfolded image dataset based on the 3D position and a result of the deformation algorithm, and
displaying, on the user interface, at least one of
an image section of the first two-dimensional unfolded image dataset or the second two-dimensional unfolded image dataset including the 2D position, or
a marking representing the 2D position in the first two-dimensional unfolded image dataset or the second two-dimensional unfolded image dataset.

14. A provision unit configured to execute the computer-implemented method as claimed in claim 1.

15. A system comprising:
a medical imaging device having a radiation source and a radiation detector; and
the provision unit as claimed in claim 14, the provision unit configured to provide a two-dimensional unfolded image dataset.

16. A non-transitory computer-readable storage medium storing computer-executable instructions that, when executed by at least one processor of a provision unit, cause the provision unit to execute the computer-implemented method of claim 1.

17. The computer-implemented method of claim 1, wherein the anatomical structure includes at least one sub-region of a hand.

18. The system of claim 15, wherein the medical imaging device is one of an X-ray imaging device or a CT device.

19. The computer-implemented method as claimed in claim 3, wherein determining of the set of characteristic specimen points includes at least one of an alignment of the two-dimensional model and the anatomical structure to one another or an adjustment of the two-dimensional model to the anatomical structure.

20. The computer-implemented method as claimed in claim 3, further comprising:
provisioning a third three-dimensional image dataset including the anatomical structure;
localizing the anatomical structure in the third three-dimensional image dataset;
determining a partial image dataset based on the third three-dimensional image dataset and the localized anatomical structure; and
provisioning the partial image dataset as the first three-dimensional image dataset.
